(19) United States
(12) Patent Application Publication
(10) Pub. No.: US 2008/0009712 A1
(43) Pub. Date: Jan. 10, 2008

(54) Apparatus and Methods for Maneuvering a Therapeutic Tool Within a Body Lumen

(76) Inventors: Mark L. Adams, Sandy, UT (US); Scott T. Bluni, Sudbury, MA (US); Paul DiCarlo, Middleboro, MA (US); Robert M. Rauker, Chester Springs, PA (US); Robert F. Rioux, Ashland, MA (US); William J. Shaw, Cambridge, MA (US); Vincent A. Turturro, Bolton, MA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 11/424,625

(22) Filed: Jun. 16, 2006

(51) Int. Cl. A61B 5/05

(52) U.S. Cl. 600/424

(57) ABSTRACT

An apparatus includes an elongate body having a plurality of discretely controllable portions. The plurality of discretely controllable portions are configured to be maneuvered within a body lumen along a predetermined path associated with an image of the body lumen. At least one portion from the plurality of discretely controllable portions may include a marker. A method includes inserting an elongate body at least partially into a body lumen. The elongate body has a plurality of discretely controllable portions. The elongate body is maneuvered within the body lumen along a predetermined path associated with an image of the body lumen. The maneuvering includes changing the relative orientation of the plurality of discretely controllable portions.

### BACKGROUND

This invention relates to medical devices and methods, and more particularly, to an apparatus and method for maneuvering a therapeutic tool within a gastrointestinal lumen.

Colorectal cancer is one of the leading causes of deaths from malignancy in the United States, with only lung cancer causing more deaths annually. Colon cancer can be prevented because it usually begins as a benign polyp that grows slowly for several years before becoming cancerous. If polyps are detected and removed, the risk of developing colon cancer is significantly reduced.

Unfortunately, widespread colorectal screening and preventive efforts are hampered by several practical impediments, including limited resources, methodologic inadequacies, and poor patient acceptance leading to poor compliance. Moreover, some tests, such as the fecal occult blood test (FOBT) fail to detect the majority of cancers and pre-cancerous polyps. Additionally, since a sigmoidoscopy only examines a portion of the colon, it also misses many polyps that occur in the remainder of the colon. The accuracy of other tests, such as the barium enema, vary and are not always reliable.

A technique for detecting colorectal cancer using helical computed tomography (CT) to create computer simulated intraluminal flights through the colon was proposed as a novel approach for detecting colorectal neoplasms by Vining D J, Shifrin R Y, Grishaw E K, Liu K, Gelfand D W, Virtual colonoscopy (Abst), Radiology Scientific Prgm 1994; 193(P):446. This technique was first described by Vining et al. in an earlier abstract by Vining D J, Gelfand D W, Noninvasive colonoscopy using helical CT scanning, 3D reconstruction, and virtual reality (Abst), SGR Scientific Program, 1994. This technique, referred to as “virtual colonoscopy” or “virtual endoscopy ”, requires, for example, a cleansed colon insufflated with air, a helical CT scan of approximately 30 seconds, and specialized three-dimensional (3D) imaging software to extract and display the mucosal surface. The resulting endoluminal images generated by the CT scan are displayed to a medical practitioner for diagnostic purposes.

There have been several advances in virtual colonoscopy that have improved the imaging techniques, making it a more viable and effective screening option. One advantage of using a virtual colonoscopy as a screening process is the reduction of the invasiveness of a traditional colonoscopy. Traditional colonoscopies are preformed using a colonoscope that has a relatively large diameter (i.e., sufficient to form a seal with the anus) that includes, among other instruments, a scope, multiple lumens for introducing gas and/or liquid, and a working channel for introducing a snare or similar device into the colon.

Another advantage of the virtual colonoscopy procedure is the elimination of the preparation process associated with a traditional colonoscopy. The typical preparation process involves the use of strong laxatives to purge any fecal waste from the colon. Such a process is extremely uncomfortable and is often cited as one of the least desirable parts of the whole procedure. Complete purging is not necessary with the virtual colonoscopy procedure. Rather, a fecal contrasting agent can be used to facilitate digital subtraction of any residual feces from the virtual image. Another advantage of the virtual colonoscopy is the reduction in the need for radiation (e.g., x-rays) when deploying devices successively.

Even though the virtual colonoscopy is largely non-invasive as a screening process, a need still exists for non-invasive and minimally invasive devices and methods for treating a gastrointestinal lumen, such as removing polyps within a colon in the event the virtual colonoscopy, or other imaging modality identifies a problem area within the colon.

### SUMMARY OF THE INVENTION

An apparatus includes an elongate body having a plurality of discretely controllable portions. The plurality of discretely controllable portions are configured to be maneuvered within a body lumen along a predetermined path associated with an image of the body lumen. At least one portion from the plurality of discretely controllable portions includes a marker. A method includes inserting an elongate body at least partially into a body lumen. The elongate body has a plurality of discretely controllable portions. The elongate body is maneuvered within the body lumen along a predetermined path associated with an image of the body lumen. The maneuvering includes changing the relative orientation of the plurality of discretely controllable portions.

### BRIEF DESCRIPTION OF THE DRAWINGS

The present invention is described with reference to the accompanying drawings.

FIG. 1 is an illustration of a large intestine.

FIGS. 2A and 2B illustrate different types of polyps in a colon.

FIG. 3 is schematic illustration of an apparatus according to an embodiment of the invention.

FIG. 4 is a schematic illustration of a system according to an embodiment of the invention.

5A is side view of an apparatus according to an embodiment of the invention, FIG. 5B is a side view of the apparatus of FIG. 5A shown with an end in a bent configuration.

FIG. 6 is a side view of an apparatus according to an embodiment of the invention.

FIG. 7 is a side view of an apparatus according to an embodiment of the invention shown partially in cross-section.

FIG. 8 is a side view of an apparatus according to an embodiment of the invention shown within a colon.

FIG. 9 is a schematic illustration of a system according to an embodiment of the invention.

FIG. 10 is a side view of a portion of a apparatus according to an embodiment of the invention shown inside a cross-sectional view of a portion of a colon.

FIG. 11 is a flowchart of a method according to an embodiment of the invention.

FIG. 12 is a flowchart of a method according to an embodiment of the invention.

### DETAILED DESCRIPTION

An apparatus includes an elongate body having a plurality of discretely controllable portions. The plurality of discretely controllable portions are configured to be maneuvered within a body lumen, such as a gastrointestinal lumen, along a predetermined path associated with an image of the body lumen. At least one portion from the plurality of discretely controllable portions includes a marker. A method includes inserting an elongate body at least partially into a body lumen. The elongate body has a plurality of discretely controllable portions. The elongate body is maneuvered within the body lumen along a predetermined path associated with an image of the body lumen. The maneuvering includes changing the relative orientation of the plurality of discretely controllable portions.

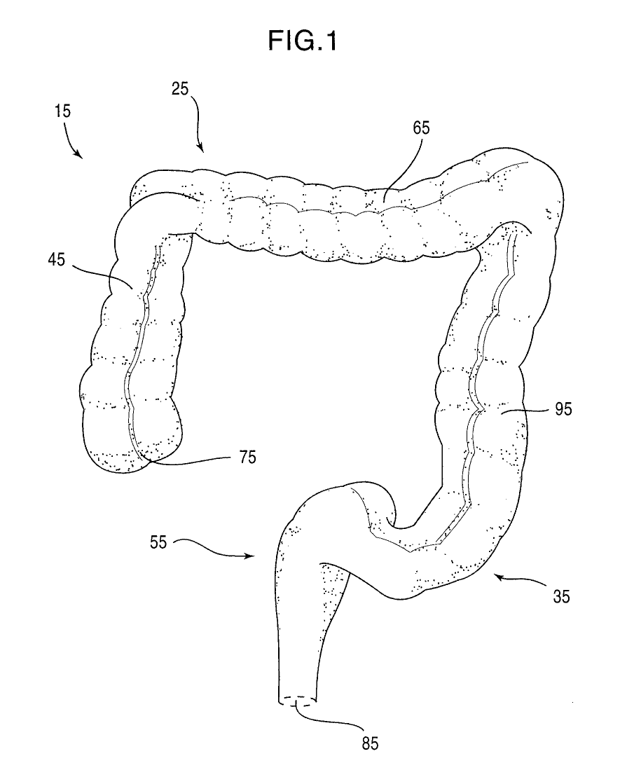

Referring to FIG. 1, an illustration of a large intestine (also called the large bowel) 15 is provided by way of background and reference. The colon 25 is the longest part of the large intestine 15, which is a tube-like organ connected to the small intestine (not illustrated) at one end, and the anus 85 at the other end. The colon 25 and the rectum 55 form the large intestine 15. The colon 25 is the first 4 to 5 feet of the large intestine 15, and the rectum 55 is the last 4 to 5 inches. The part of the colon 25 that joins to the rectum 55 is called the sigmoid colon 35. The junction of the two parts is often referred to as the rectosigmoid colon or rectosigmoid process. The part of the colon 25 that joins to the small intestine is called the cecum 75. The cecum 75 is adjacent the ascending colon 45, which is connected to the transverse colon 65. The transverse colon 65 is connected to the descending colon 95, which is connected to the sigmoid colon 35. The colon 25 removes/absorbs water and some nutrients and electrolytes from partially digested food. The remaining material, solid waste, called stool or feces, moves through the colon 25 to the rectum 55 and leaves the body through the anus 85.

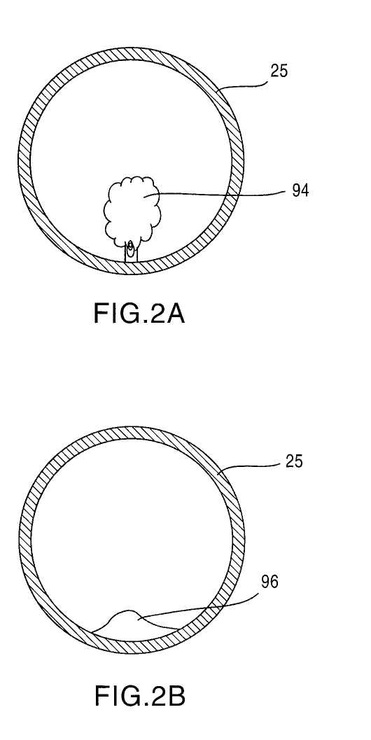

FIGS. 2A-2B illustrate various types of polyps that can form in the colon. A gastrointestinal polyp is a mass of the mucosal surface of the intestine that protrudes into the passageway of the bowel. Polyps can be neoplastic, non-neoplastic, or submucosal. Adenomatous polyps are abnormal growths in the colon and are more likely to develop into or already contain cancer than other types of colon polyps. Adenomatous polyps, however, usually contain tissue that is abnormal but not necessarily cancerous, hence the importance of being able to completely remove a polyp from the colon. The size, type of tissue, and degree of abnormality (mild, moderate, or severe) in a polyp determines the likelihood that it contains cancer.

Some adenomatous polyps are attached to the wall of the colon 25 (or rectum) by a stalk (a pedunculated polyp 94) as illustrated in FIG. 2A. Some polyps have a broad base with little or no stalk (a sessile polyp 96) as illustrated in FIG. 2B.

The apparatuses, systems and methods of the present invention involve the use of an apparatus in conjunction with known imaging devices, including virtual imaging modalities, and a processor, to maneuver the apparatus through a body lumen, such as a colon. Although the below description focuses primarily on medical treatments within a colon, such as removal of a polyp from a colon, the methods and medical procedures described can be used in other body lumens, such as other gastrointestinal lumens including, for example, the esophagus, stomach, and small intestine.

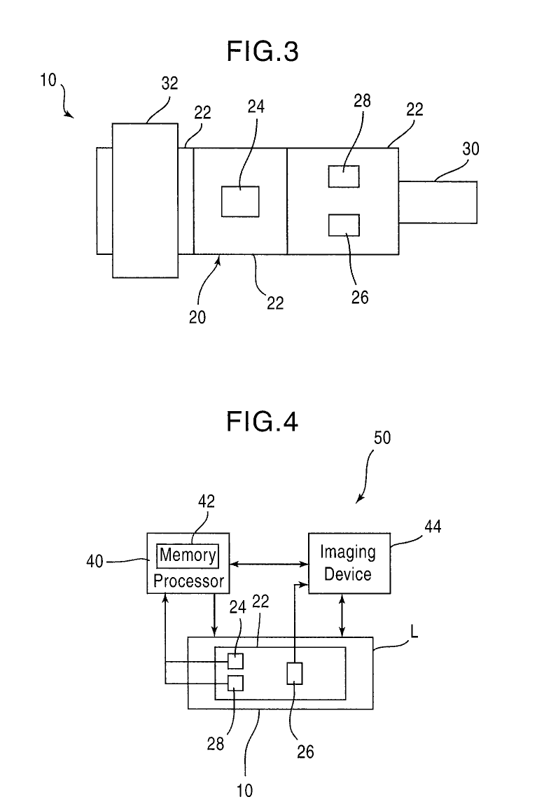

A schematic illustration of an apparatus 10 is shown in FIG. 3. The apparatus 10 includes an elongate body 20 having a plurality of portions 22. The portions 22 can be discretely and remotely controlled to help maneuver the apparatus 10 through a gastrointestinal lumen, such as a colon. The portions 22 can be for example, piezo electric elements that can be caused to move based on an input of voltage, which will be discussed in more detail below. The elongate body 20 can also include one or more flexible members (not shown in FIG. 3) coupled to one or more of the portions 22. The flexible member can be used to help maneuver the elongate body 20 through a gastrointestinal lumen. The apparatus 10 can also include at least one proximity sensor 24, at least one marker 26, and at least one orientation sensor 28 coupled to one or more of the plurality of portions 22.

The apparatus 10 can include a medical tool 30 and an expandable member 32, such as an inflatable balloon, each coupled to the elongate body 20. In some embodiments, the medical tool 30 can be configured to be moveably disposed within a lumen (not shown in FIG. 3) defined by the elongate body 20. In other embodiments, the medical device 30 can be coupled to an end of the elongate body 20, or can be coupled to the elongate body 20 such that the medical tool 30 is positioned proximate the elongate body 20 in a side-by-side relationship. The medical tool 30 and the elongate body 20 can also be monolithically formed. The medical tool 30 can be a variety of different medical devices including, for example, a snare, graspers, forceps, an endoscope, etc.

The expandable member 32 can be coupled to the elongate body 20 such that the expandable member 32 surrounds at least a portion of an exterior surface of the elongate body 20. The expandable member 32 can be used to prevent the elongate body 20 from contacting the interior walls of a gastrointestinal lumen.

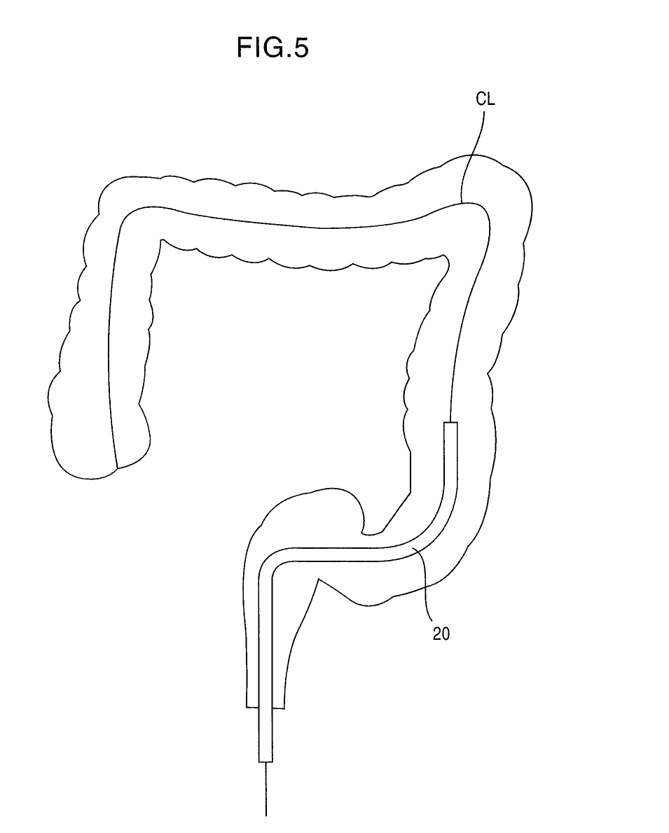

The apparatus 10 can be used in conjunction with other devices, such as a processor 40 and an imaging device 44, as shown in FIG. 4. FIG. 4 is a schematic illustration of a system 50 and illustrates a single portion 22 of the apparatus 10 (for simplicity) coupled to, or in communication with, the processor 40 and the imaging device 44. The processor 40 can be, for example, a commercially available personal computer, or a less complex computing or processing device that is dedicated to performing one or more specific tasks. The processor 40, according to one or more embodiments of the invention, can be a commercially available microprocessor. Alternatively, the processor 40 can be an application-specific integrated circuit (ASIC) or a combination of ASICs, which are designed to achieve one or more specific functions, or enable one or more specific devices or applications. In yet another embodiment, the processor 40 can be an analog or digital circuit, or a combination of multiple circuits.

The processor 40 can include a memory component 42. The memory component 42 can include one or more types of memory. The processor 40 can store data in the memory component 42 or retrieve data previously stored in the memory component 42. The components of the processor 40 can communicate with devices external to the processor 40 by way of an input/output (I/O) component (not shown), or communicate remotely, via radio waves, for example. According to one or more embodiments of the invention, the I/O component can include a variety of suitable communication interfaces.

The imaging device 44 can include a variety of different imaging modalities, such as a computed tomography (CT) device, a magnetic resonance imaging (MRI) device, an ultrasound device or infrared tracking device. A virtual imaging modality can also be used, such as a virtual colonoscopy, or any other type of non-invasive imaging modality. The imaging device 44 can be in communication with the processor 40, and send, transfer or otherwise provide imaging data to the processor 40. Software configured to be used with virtual endoscopy can also be used. The imaging data can include, for example, an image of a gastrointestinal lumen. The processor 40 can use the image data to assist in accurately maneuvering the elongate body 20 through the gastrointestinal lumen, which is described in more detail below.

As illustrated in FIG. 4, the proximity sensor 24 can be in communication with the processor 40 and used to identify the location of the elongate body 20 relative to an interior wall of a gastrointestinal lumen L. For example, the proximity sensor 24 can send a signal to the processor 40, which receives and stores the sensor proximity signal while the elongate body 20 is maneuvered through the gastrointestinal lumen L. The marker 26 is configured to be visible on imaging device 44. For example, the marker 26 can be, for example, a radiopaque marker such as those used with a CT scan, x-rays, or fluoroscope, having a density of about 9.9 g/cc or greater. Some examples of materials of such markers and their density, include tantalum (16.6 g/cc), tungsten (19.3 g/cc), rhenium (21.2 g/cc), bismuth (9.9 g/cc), silver (16.49 g/cc), gold (19.3 g/cc), platinum (21.45 g/cc), and iridium (22.4 g/cc). Other radiopaque markers include bismuth oxychloride, bismuth trioxide, and tungsten oxide.

Marker 26 can also be a MRI visible marker, such as non-ferrous metal-alloys containing paramagnetic elements (e.g., dysprosium or gadolinium) such as terbium-dysprosium, dysprosium, and gadolinium; non-ferrous metallic bands coated with an oxide or a carbide layer of dysprosium or gadolinium (e.g., Dy₂O₃ or Gd₂O₃); non-ferrous metals (e.g., copper, silver, platinum, or gold) coated with a layer of superparamagnetic material, such as nanocrystalline Fe₃O₄, CoFe₂O₄, MnFe₂O₄, or MgFe₂O₄; and nanocrystalline particles of the transition metal oxides (e.g., oxides of Fe, Co, Ni). Powder of MRI visible materials can be mixed with the material of the embolic particles, e.g., shape memory polymer.

Marker 26 used with an ultrasound device can be, for example, grooved, knurled, threaded metallic bands or members such as stainless steel, void filled polymers or ceramic bands or members

Because the marker 26 can be viewed on the imaging device 44, the marker 26 can be used to visually identify the location of a particular portion 22 of the elongate body 20 within the gastrointestinal lumen L. The imaging device can communicate marker 26 location information to the processor 40.

The orientation sensor 28 can be, for example, a sensor configured to output a signal associated with the orientation of at least one of the portions 22 of the elongate body 20 to the processor 40. In response to an orientation signal received at the processor 40, the processor 40 can send a control signal to one or more of the portions 22 to cause it to move in a desired direction within the gastrointestinal lumen. The signal can be, for example, a voltage signal sent via a flexible member coupled to the portions 22. Alternatively, the signal can be a remotely detectable signal, such as, for example, a radio frequency identification (RFID) signal, which is sent via a wireless connection to the portions 22.

In one use of the apparatus 10 and/or the system 50, an image of a patient's colon (or other gastrointestinal lumen) is taken by the imaging device 44. The image data can be viewed on the imaging device 44 or transferred to the processor 40 and viewed. The image data can identify a polyp, tumor, cyst or other area of interest within the gastrointestinal lumen. The processor 40 can use the image data to determine a center line CL of the gastrointestinal lumen. As shown in FIG. 5, the elongate body 20 can then be inserted into the gastrointestinal lumen, and maneuvered to a desired area of interest using the center line CL as a guide. The elongate body 20 can be maneuvered manually or automatically depending on the particular embodiment. The elongate body 20 may have a medical tool coupled thereto, to be used to treat the area of interest.

With the elongate body 20 positioned within the gastrointestinal lumen, the imaging device can further image the gastrointestinal lumen. The marker 26 can then be viewed on the image data to determine a location of the elongate body within the gastrointestinal lumen at any given time. As stated previously, the proximity sensor 24 and the orientation sensor 28 can each send signals to the processor 40 to assist with directing the elongate body 20 through the gastrointestinal lumen. For example, when the proximity sensor 24 data indicates that a portion 22 is coming near an interior wall of the lumen, for example at a turn in the lumen, the processor 40 can send a control signal to that portion 22 to cause it to re-orient or bend so as to avoid contacting the interior wall of the lumen. Likewise, the orientation sensor 28 can send signals to the processor 40 indicating the orientation of a portion 22 within the gastrointestinal lumen at a given time.

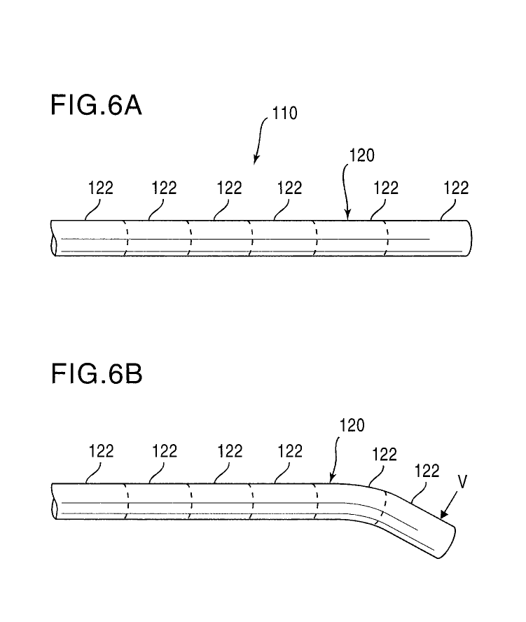

FIGS. 6A and 6B illustrate a portion of an apparatus 110 according to an embodiment of the invention. Apparatus 110 includes an elongate body 120. FIG. 6A shows the elongate body 120 in a straight configuration, and FIG. 6B illustrates a portion 122 of elongate body 120 in a bent configuration after receiving a control signal, such as applied voltage V from a processor. Although only one portion 122 is illustrated as being bent, the elongate body 120 can include multiple portions 122 that are also discretely controllable by a processor.

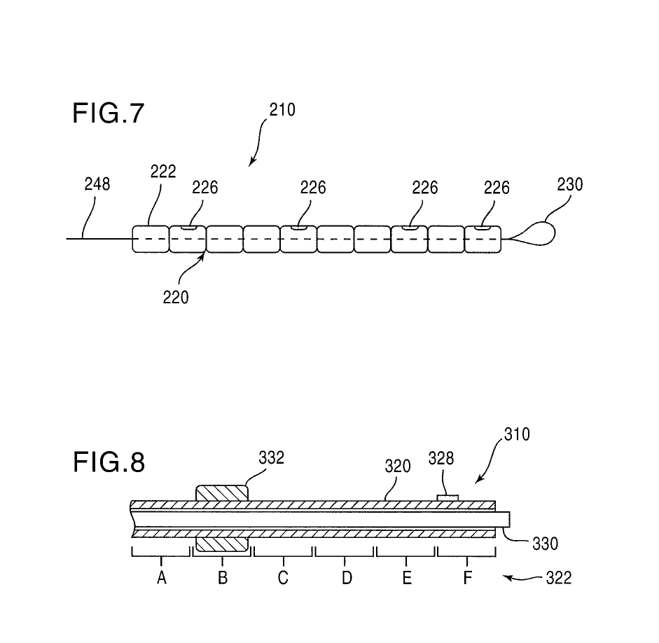

FIG. 7 illustrates an apparatus according to another embodiment of the invention. An apparatus 210 includes an elongate body 220 having a plurality of discretely controllable portions 222 and a flexible member 248. A medical tool 230 in the form of a snare is coupled to an end of the elongate body 220. In this embodiment, there are four markers 226 placed on separate portions 222. The flexible member 248 can be coupled to a device (not shown) configured to provide a source of applied voltage to selected portions 222 to maneuver the elongate body 220 within a body lumen as described above. The apparatus 210 can also include at least one proximity sensor (not shown in FIG. 7), and at least one orientation sensor (not shown in FIG. 7) coupled to one or more of the portions 222 that can function in the same manner as described in the previous embodiment. The flexible member 248 can also include multiple wires, each coupled to a selected portion 222 to conduct electricity to that portion 222.

FIG. 8 illustrates an apparatus according to yet another embodiment of the invention. An apparatus 310 includes an elongate body 320 having a plurality of discretely controllable portions 322 (labeled as A-F). Although the elongate body 320 appears to be one continuous component, the portions 322 can be separately moved or re-oriented. In this embodiment, an expandable member 332 is coupled to the elongate body 320 and an orientation sensor 328 is coupled to portion 322 F. Although only one orientation sensor 328 is illustrated, more can be included. In addition, the apparatus 310 can include one or more proximity sensors (not shown in FIG. 8). A medical tool 330, such as an endoscope, is movably disposed within a lumen defined by the elongate body 320.

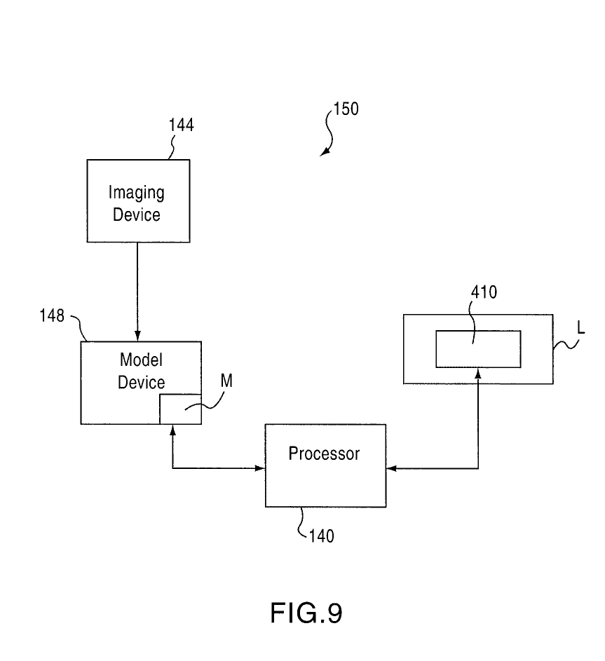

In another embodiment illustrated in FIG. 9, a system 150 includes a model device 148 configured to create a model of a body lumen, such as a model of a colon or other gastrointestinal lumen, based on image data associated with the body lumen. The model device 148 can include a processor 140 as described above in FIG. 4. The model device 148 can be in communication with an imaging device 144. In this embodiment, the imaging device 144 can take one or more images of the body lumen and provide the image data to the model device 148. The model device 148 then creates a clear model M, constructed for example of a clear plastic material, of the body lumen that can be used to help maneuver the elongate body 20 through the body lumen. The model M can be used in conjunction with a control element (not shown in FIG. 9) positionable within the model. In some embodiments, the control element is in the form of a wire, in other embodiments, the control element can include other shapes and configurations.

The control element can be in communication with a processor 140 and the processor 140 can be in communication with an apparatus 410. The apparatus 410 can include an elongate body having a plurality of discretely controllable portions, as described above. The processor 140 can send signals to the portions of the elongate body of apparatus 410 as described above, but the signals in this embodiment are based on the maneuvering of the control element through the model M of the body lumen. For example, a physician or other health care professional can maneuver the control element through the model M. Because the model M is clear, the physician can visually see the direction in which to maneuver the control element through the lumen. The control element is configured to communicate its location in the body lumen model to the processor 140, which in turn simultaneously controls the movement of the elongate body of apparatus 410 through the actual body lumen. In some embodiments, a virtual body lumen, such as a virtual colon or other gastrointestinal lumen can be viewed on a graphical user interface. A control element can be moved through the virtual computer model, which in turn provides control signals to maneuver an elongate body through the actual body lumen.

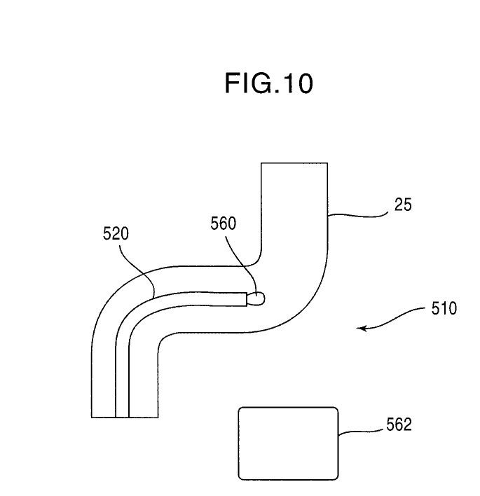

In another embodiment, illustrated in FIG. 10, an apparatus 510 includes an elongate body 520 in the form of a flexible wire or tube. A magnet element 560 is disposed on the elongate body 520. An external robotic magnet 562 (shown schematically) is configured to direct the magnet element 560 through the colon 25. For example, a robot arm can be mounted to a CT or MRI table and the robot arm can have a magnet disposed thereon. The path of the robot arm can be guided by the CT or MRI scan. The robot arm can follow the body lumen externally, and the magnetic field between the external robot magnet and the magnet element 560 on the elongate body 520 causes the elongate body 520 to follow the path of the external robot magnet. The robot arm can also include one or more proximity sensors. In some embodiments, electromagnets can be used.

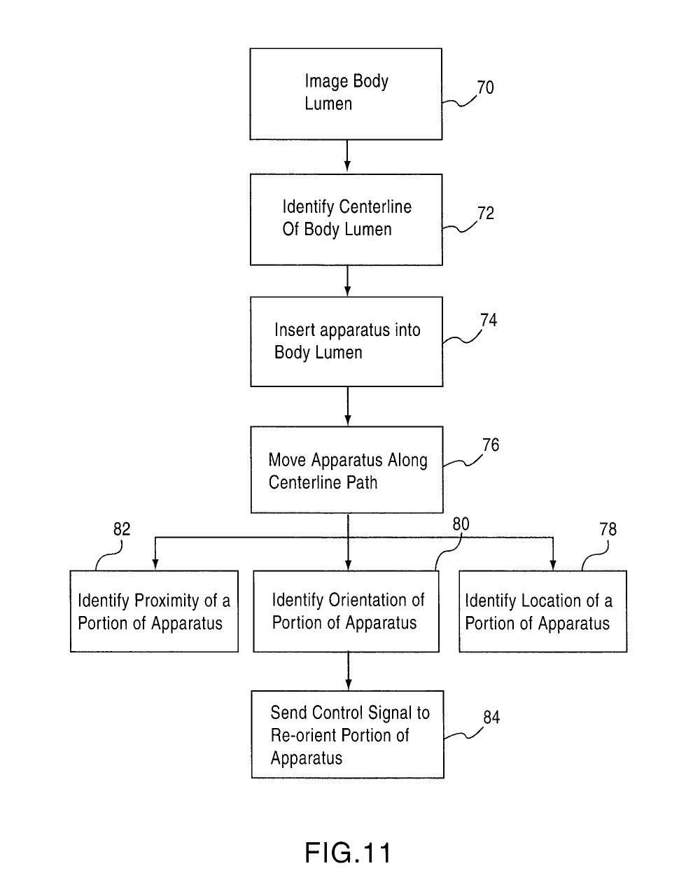

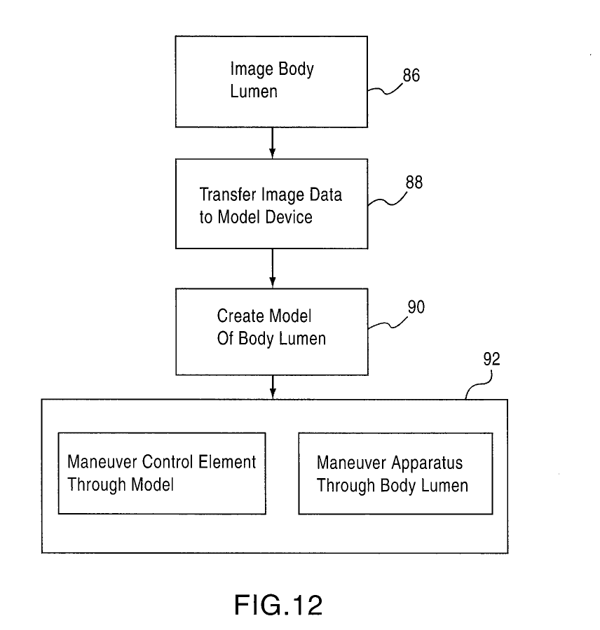

A method according to an embodiment of the invention is illustrated in the flowchart of FIG. 11. The method includes at 70, imaging a body lumen, such as a colon. The image data is transferred to a processor where a centerline of the lumen is determined based on the image data at 72. An apparatus according to the invention is inserted into the body lumen at 74. At 76, the apparatus is moved along the centerline path identified at 72. At s78 a location of a portion of the apparatus within the body lumen can be identified using a marker coupled to the portion and visible on an image. An orientation of a portion of the apparatus can be identified via a sensor coupled to the portion that sends a signal to the processor at 80. At 82, the orientation of a portion of the apparatus can be modified or re-oriented by sending a signal from the processor to the portion. The proximity of a portion of the apparatus can be identified at 84. Steps 80-84 can all be performed, or depending on the particular configuration of the apparatus, only some of these steps may be performed. The apparatus can be advanced further along the centerline path by repeating steps 76 through 84.

In another embodiment, a method includes imaging a body lumen, such as a gastrointestinal lumen at 86. The image data is transferred to a model device at 88. The model device can make a clear model of the body lumen based on the image data at 90. Alternatively, the model can be a virtual computer model. A control element in communication with a processor is maneuvered through the model at 92, while at the same time an apparatus having discretely controllable portions is maneuvered through the actual body lumen based on the movement of the control element through the clear model.

### CONCLUSION

While various embodiments of the invention have been described above, it should be understood that they have been presented by way of example only, and not limitation. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

For example, the discretely controllable elements can alternatively include shapeable elements formed with shape-memory materials, such as certain polymers and metals, that can be individually controlled. The discretely controllable elements can also be in the form of individually controlled pull wires, or individually controllable inflatable portions. In some embodiments, an apparatus according to the invention can be in the form of a guide wire with controllable portions, rather than an elongate body defining a lumen. The guide wire can include discretely controllable portions as described herein and be couplable to another medical device such as a snare or endoscope.

What is claimed is:

1. An apparatus, comprising:
an elongate body including a plurality of discretely controllable portions, the plurality of discretely controllable portions configured to be maneuvered within a body lumen along a predetermined path associated with an image of the body lumen; and
at least one portion from the plurality of discretely controllable portions including a marker.

2. The apparatus of claim 1, further comprising:
a sensor disposed on at least one portion from the plurality of discretely controllable portions, the sensor configured to output a signal associated with an orientation of the at least one portion on which the sensor is disposed.

3. The apparatus of claim 1, further comprising:
a sensor disposed on at least one portion from the plurality of discretely controllable portions, the sensor configured to output an orientation signal associated with an orientation of the at least one portion on which the sensor is disposed to a processor, the processor being configured to output a control signal to at least one portion of the plurality of discretely controllable portions.

4. The apparatus of claim 1, wherein each portion of the plurality of discretely controllable portions is configured to move based on a control signal provided to the discretely controllable portion.

5. The apparatus of claim 1, wherein the marker is configured to indicate a location within the gastrointestinal lumen of the at least one portion from the plurality of discretely controllable portions.

6. The apparatus of claim 1, further comprising:
a model device in communication with the elongate body and configured to influence the movement of the elongate body within the body lumen.

7. The apparatus of claim 1, further comprising:
a proximity sensor coupled to at least one portion from the plurality of discretely controllable portions, the proximity sensor configured to output a proximity signal associated with a distance from an interior wall of the gastrointestinal lumen.

8. The apparatus of claim 1, further comprising:
at least one expandable member coupled to the elongate body, the at least one expandable member configured to engage, and to prevent the elongate body from contacting, an interior wall of the body lumen.

9. The apparatus of claim 1, wherein the elongate body includes a flexible element and the plurality of discretely controllable portions are coupled to the flexible element.

10. The apparatus of claim 1, wherein the predetermined path is substantially defined by a centerline of the body lumen.

11. The apparatus of claim 1, wherein each portion from the plurality of discretely controllable portions is configured to be re-oriented based on the location of the discretely controllable portion within the body lumen.

12. The apparatus of claim 1, wherein the body lumen is a gastrointestinal lumen.

13. The apparatus of claim 1, further comprising:
an actuator in communication with the elongate body configured to cause movement of at least one portion from the plurality of discretely controllable portions

14. A method, comprising:
inserting an elongate body at least partially into a body lumen, the elongate body having a plurality of discretely controllable portions; and
maneuvering the elongate body within the body lumen along a predetermined path associated with an image of the gastrointestinal lumen including changing the relative orientation of the plurality of discretely controllable portions.

15. The method of claim 14, further comprising:
imaging the body lumen prior to insertion of the elongate body.

16. The method of claim 14, wherein the inserting includes inserting the elongate body into the body lumen at a predetermined rate of insertion.

17. The method of claim 14, wherein the predetermined path is substantially along a centerline of the body lumen.

18. The method of claim 14, further comprising:
providing a control signal to a selected one from the plurality of discretely controllable portions to cause a change in the relative orientation of the plurality of discretely controllable portions.

19. The method of claim 14, further comprising:
outputting a signal associated with an orientation of one portion from the plurality of discretely controllable portions.

20. The method of claim 14, further comprising:
identifying a location within the body lumen of at least one portion from the plurality of discretely controllable portions.

21. The method of claim 14, wherein the body lumen is a gastrointestinal lumen.

22. A processor-readable medium storing code representing instructions to cause a processor to perform a process, the code comprising code to:
insert an elongate body at least partially into a body lumen, the elongate body having a plurality of discretely controllable portions; and
maneuver the elongate body within the body lumen along a predetermined path associated with an image of the body lumen including changing the relative orientation of the plurality of discretely controllable portions.

23. The processor-readable medium of claim 22, further comprising code to:
image the body lumen prior to insertion of the elongate body.

24. The processor-readable medium of claim 22, further comprising code to:
insert the elongate body into the body lumen at a predetermined rate of insertion.

25. The processor-readable medium of claim 22, further comprising code to:
maneuver the elongate body within the body lumen along a predetermined path substantially defined by a centerline of the body lumen.

26. The processor-readable medium of claim 22, further comprising code to:
provide a control signal to a selected one of the plurality of discretely controllable portions to cause a change in the relative orientation of the plurality of discretely controllable portions.

27. The processor-readable medium of claim 22, further comprising code to:
receive a signal associated with an orientation of one portion from the plurality of discretely controllable portions.

28. The processor-readable medium of claim 22, further comprising code to:
identify a location within the body lumen of at least one portion from the plurality of discretely controllable portions.

29. An apparatus, comprising:
a plurality of discretely controllable elements configured to be inserted into a body lumen and maneuvered along a predefined path within the body lumen, the plurality of discretely controllable elements each including a sensor and a marker, the sensor configured to output a signal associated with an orientation of the plurality of discretely controllable elements, the marker configured to indicate a location of the plurality of discretely controllable elements within the gastrointestinal lumen.

30. The apparatus of claim 29, wherein a selected one of the plurality of discretely controllable elements is configured to move in response to a control signal, the control signal being based on the identified location.

31. The apparatus of claim 29, wherein the predetermined path is defined by a centerline of the body lumen.

32. The apparatus of claim 29, wherein the body lumen is a gastrointestinal lumen.

33. The apparatus of claim 29, wherein the body lumen is a colon.
